(19) United States
(12) Patent Application Publication
(10) Pub. No.: US 2026/0248657 A1
(43) Pub. Date: Aug. 27, 2026

(54) MANUFACTURING DEVICE FOR GENERATING CLOSURE TABS FOR A DIAPER

(71) Applicant: Gottlieb Binder GmbH & Co. KG, Holzgerlingen (DE)

(72) Inventors: Konstantinos POULAKIS, Hildrizhausen (DE); Clovis NKUIDJEU, Neustetten (DE); Lars HEEPE, Tübingen (DE)

(73) Assignee: Gottlieb Binder GmbH & Co. KG, Holzgerlingen (DE)

(21) Appl. No.: 19/647,874

(22) Filed: Apr. 15, 2026

Related U.S. Application Data
(63) Continuation of application No. PCT/EP2024/076771, filed on Sep. 24, 2024.

(30) Foreign Application Priority Data
Oct. 17, 2023 (DE) 102023210156.4

(51) Int. Cl. A61F 13/15

(52) U.S. Cl. CPC A61F 13/15585; A61F 2013/15861

(57) ABSTRACT

A production method for producing a closure tab for a diaper has the method steps of: a) providing a hook strip and at least one textile or textile-like backing layer strip; b) placing the hook strip and the backing layer strip against one another and fastening the hook strip to the backing layer strip at least in portions; c) cutting at least one hook portion, preferably multiple thereof, out of the hook strip; d) producing a closure tab strip by detaching the hook strip from the at least one backing layer strip. The at least one cut-out hook portion remains on the backing layer strip.

This continuation application claims priority to PCT/EP2024/076771 filed on September 24, 2024 which has published as WO 2025/082707 A1 and also the German application number DE 10 2023 210 156.4 filed on October 17, 2023, the entire contents of which are fully incorporated herein with these references.

### DESCRIPTION

### FIELD OF THE INVENTION

The invention relates to a production method for producing closure tabs for a diaper. Furthermore, the invention relates to a production device for producing closure tabs. Furthermore, the invention relates to a diaper comprising closure tabs.

### BACKGROUND OF THE INVENTION

Various types of diapers, in particular disposable or single-use diapers, are known from the prior art. So-called diaper pants or pull-on diapers are designed in the form of a trouser-like, closed shape and can be put on by a human wearer like pants, with the legs passing through the diaper. This type of diaper typically has multiple predetermined tear points that allow the diaper to be torn open to take it off. This allows the used diaper to be hygienically removed from the wearer without the legs coming into contact with the contaminated diaper.

Another type of diaper involves the use of closure tabs used for closing the diaper. Typically, this type of diaper is placed around the crotch of a human wearer without the closure tabs closed and then fixed in the pelvic region by means of the closure tabs, ensuring that the diaper is held securely on the wearer. Typically, when fastened, the fastening tabs form a hook-and-loop connection that can be released to remove the diaper. Once the diaper has been removed, the hook-and-loop connection can then be used again to secure the rolled-up diaper, thus preventing the region around the used diaper from being contaminated thereby.

Diapers and methods for producing closure tabs are known from EP 1 941 853 B1 and EP 2 641 574 B1. The methods described are optimized for the greatest possible material utilization, which limits the flexibility of production to just a few diaper types and diaper sizes.

US 6,730,069 B2 discloses a production method for producing mechanical connectors, in which molten polymer is poured into recesses in a backing strip so as to subsequently form hook-and-loop connectors from the molten polymer. However, the fasteners designed in this way have a reduced material thickness of the backing layer in the region of the hook-and-loop fasteners, which reduces strength.

In addition, the known methods are technically demanding to implement, and the production devices used are prone to error, leading to production downtimes.

### SUMMARY OF THE INVENTION

### Object of the invention:

The object of the invention is therefore to specify a production method with which various types of closure tabs can be produced cost-effectively in a simple, fast and robust manner. Furthermore, the object of the invention is to specify a production device and a diaper comprising a closure tab.

### Description of the invention:

This object is achieved according to the invention by a production method having the features of claim 1. The object is also achieved by a production device having the features of claim 5. Furthermore, the object is achieved by a diaper according to claim 9. The dependent claims describe preferred embodiments of the invention.

According to the invention, a production method is provided. The production method is designed to produce a closure tab. The closure tab is typically suitable for closing a diaper as described above and below.

The production method has at least the following method steps:

A first method step a) provides the provision of a hook strip and at least one textile or textile-like backing layer strip. The hook strip and the at least one backing layer strip are preferably provided as rolls. This simplifies further processing.

The hook strip here and in the following can be understood to mean a hook-and-loop strip having a plurality of hooks and/or mushrooms (mushroom-shaped hooks/elements). The hook strip is typically produced in a separate process that takes place prior to the production method. Preferably, the hook strip is made in one piece and/or from a single material.

The hook-and-loop strip preferably has a hook strip width of at least 2 millimeters, particularly preferably at least 5 millimeters. The hook-and-loop strip can preferably have a hook strip width of at most 20 millimeters, particularly preferably at most 10 millimeters. In a specific design, the hook strip has between 50 and 5000 hooks per square centimeter. The hooks are typically arranged in rectangles, hexagons and/or randomly. The aforementioned properties of the hook strip have proven to be particularly suitable, individually and in combination, for forming a hook-and-loop connection on a diaper.

The backing layer strip is typically understood to mean a strip with minimal material thickness, limited width and extended length. In other words, the backing layer strip can substantially be understood as a two-dimensional sheet material.

The textile backing layer strip can be understood here and in the following to mean a strip made from a spinnable, weavable and/or knittable material. Preferably, the textile backing layer strip has nonwoven material and/or is made of nonwoven material.

A textile-like backing layer strip can be understood here and in the following to mean a strip made from a non-textile material having similar properties to a textile strip. Preferably, the textile-like backing layer strip is designed as a film. Further preferably, the textile-like strip has or is formed from a thermoplastic, particularly preferably a polyolefin, in particular, a polypropylene.

Additional method step b) provides placing the hook strip on the backing layer strip, in particular so as to be surface-to-surface. In other words, the method creates surface contact between the hook strip and the backing layer strip. The surface contact is preferably created over the entire width of the hook strip, ensuring it is placed on the backing layer strip without any wrinkles. In addition, the hook strip is fastened to the backing layer strip, at least in portions. “Attached in portions” can be understood to mean that it is fastened at specific points or over a limited surface region. In other words, when the hook strip is fastened in portions, it is not fastened to the entire surface of the backing layer strip. This can simplify the subsequent method steps. Attaching said hook strip can prevent any relative movement between the hook strip and the backing layer strip.

To attach the hook strip to the backing layer strip, a binder, in particular, an adhesive, is preferably introduced into the contact region between the hook strip and the backing layer strip. The binder is particularly preferably introduced immediately before the hook strip and backing layer strip are placed against one another, which provides particularly secure attachment and allows the use of a binder that hardens particularly quickly. Alternatively or additionally, the hook strip can be welded to the backing layer strip. Welding enables the hook strip to be fastened particularly precisely and quickly. Welding also enables the hook strip to be fastened to the backing layer strip when placed thereon.

Alternatively or additionally, attachment in portions can involve the hook strip and/or the backing layer strip already having an adhesive surface in the provided state. This allows the hook strip to be fixed and/or fastened to the entire surface of the backing layer strip.

Preferably, a rubber-based and/or polyolefin-based binder is used for bonding. This ensures that the hook strip is fastened in a manner that is well tolerated by the skin.

Further preferably, welding is carried out by ultrasonic or high frequency welding. This allows the hook strip to be fastened without damaging it and/or the backing layer strip.

In additional method step c), at least one, preferably multiple, hook portions is/are cut out of the hook strip. This is preferably done by undoing the mechanical connection or the material cohesion between the hook strip and the corresponding hook portion. Typically, this is done by cutting the hook strip along a designated edge of the hook portion to be formed.

According to the invention, “cutting out” can be understood to mean a weakening of the material strength between the hook strip and the hook portion to be formed, wherein the retaining forces between the hook strip and the hook portion are then lower than the retaining forces between the backing layer strip and the hook portion. By attaching the hook strip in the region of the hook portions to be cut out, the retaining forces between the hook portion and the backing layer strip can be greatly increased, whereby the hook portions can also be cut out in the event that the material strength is reduced to a lesser extent. Weakening the material strength advantageously prevents damage to the backing layer strip when cutting the hook portions out of the hook strip attached to the backing layer strip.

Furthermore, method step d) is provided in which a closure tab strip is produced by detaching the hook strip from the at least one backing layer strip. When the hook strip is detached from the backing layer strip, the at least one cut-out hook portion remains on the backing layer strip. In other words, the hook strip that was previously attached to the backing layer strip is detached therefrom again. Since the retaining forces between the hook portion and the backing layer strip exceed the retaining forces between the hook portion and the hook strip, the hook portion remains on the backing layer strip as the hook strip is detached.

The closure tab strip produced thus at least has the one backing layer strip and at least one hook portion detached from the hook strip, wherein the hook portion is fastened to the backing layer strip. Preferably, the closure tab strip has multiple, in particular a large number of, hook portions arranged at a distance from one another on the backing layer strip.

In summary, the production method according to the invention provides for attaching hook portions of a hook strip to a backing layer strip, wherein the hook portions are formed during or after the hook strip has been placed against the backing layer strip. The described method thus proposes the creation of a closure tab strip using simple means and method steps, thereby minimizing the robustness of the production method as well as the provision and maintenance costs. In addition, the method is particularly suitable for implementation as a continuous process where the hook strip and the backing layer strip are continuously fed for further processing, which can increase production speed and further reduce production costs.

In a preferred embodiment of the production method, in method step a) a further backing layer strip is provided and in method step c) the backing layer strip is fastened to the further backing layer strip. In other words, the production method can be carried out by providing at least two backing layer strips. This makes it particularly easy to adapt the production method to different dimensions of closure tabs to be produced using the same hook strip and the same backing layer strip. In addition, the strength of the closure tabs can be increased as a result of a multi-layer design of the closure tab strip.

The further backing layer strip is preferably fastened to the backing layer strip by introducing a binder, in particular an adhesive, into the contact region between the backing layer strip and the further backing layer strip. Preferably, the further backing layer strip is fastened to the entire surface of the backing layer strip. The binder is preferably introduced immediately before the backing layer strip is placed against the further backing layer strip. Alternatively or additionally, the backing layer strip and/or the further backing layer strip has/have an adhesive surface which is designed to fasten the further backing layer strip to the backing layer strip.

In a preferred embodiment, the production method has additional method step e), which provides for the production of closure tabs by forming closure tab edges by removing a portion of the closure tab strip. In other words, the closure tab strip is separated or divided into closure tabs. The cutting of the closure tab strip is preferably carried out by cutting into or cutting through it. The closure tab edge is formed at a predetermined minimum distance from the at least one hook portion. By setting a hook portion back with respect to the edge of the closure tab, the closure tab can be prevented from having hard and/or sharp edges. As a result, the wear comfort of the diaper can be increased by using the closure tabs and reduce the risk of injury, such as cuts, when putting on and taking off the diaper.

In one specific embodiment, the minimum distance is 1 or more millimeters, preferably2 or more millimeters, particularly preferably 3 or more millimeters.

Particularly preferred is an embodiment of the production method in which method step b) and method step c) are carried out simultaneously. In other words, the hook strip, the backing layer strip and, if applicable, the further backing layer strip are placed against one another whilst, or at the same time as, the hook portions are cut out of the hook strip. This can increase the method duration or production speed, thereby increasing productivity and minimizing costs.

The object addressed by the invention is also achieved by a production device.

The production device is designed to produce a closure tab, in particular for a diaper. The production device is particularly designed to carry out the production method described above and below.

The production device has a supply unit. The supply unit is designed to provide a hook strip and at least one backing layer strip. Preferably, the supply unit has a magazine, in particular a reel magazine. The reel magazine can have reels for providing the strips, in particular a first supply reel for providing the hook strip and a second supply reel for holding the backing layer strip.

The production device also has a joining unit for joining the hook strip to the backing layer strip. The joining unit is typically designed to place the hook strip against the backing layer strip. In addition, the joining unit is designed to cut the at least one hook portion, in particular, a plurality of hook portions, out of the hook strip.

In addition, the production device has a removal unit. The removal unit is designed to receive the hook strip and the closure tab strip separately. Preferably, the removal unit has a magazine, in particular, a reel magazine having multiple reels for separately receiving the strips. Further preferably, the removal unit has a first removal reel for receiving the hook strip and a second removal reel for receiving the closure tab strip.

The joining unit preferably has a drive unit. Preferably, the drive unit is designed to drive the supply unit, the joining unit and/or the removal unit.

During operation of the production device, the hook strip and the backing layer strip are typically conveyed from the supply unit, in particular, by unwinding the supply reels, into the joining unit. Within the joining unit, the hook strip and the backing layer strip are placed against one another, in particular fastened to one another. During this time or thereafter, the hook portions are cut out of the hook strip. The hook strip is then deflected relative to the backing layer strip, causing the hook strip to detach from the backing layer strip inside the joining unit and thus producing the closure tab strip. The hook strip can then be received in the removal unit separately from the closure tab strip. Preferably, the hook strip is detached from the backing layer strip by appropriately positioning the removal device relative to the joining unit such that said strips are received by the removal device in such a way that the hook strip is locally spaced apart from the backing layer strip. Furthermore, the hook strip may be locally spaced from the backing layer strip by providing deflection devices, in particular deflection reels.

In summary, the invention proposes a production device having a particularly simple structure. This allows provision costs and maintenance effort to be minimized. In addition, the production device enables the production method to be carried out with particularly high process reliability.

In a preferred embodiment of the production device, the joining unit comprises reel tools between which the hook strip and the backing layer strip are guided. A particularly high production speed can be achieved by means of reel tools. The reel tools are designed and arranged relative to one another for placing, in particular, pressing, the hook strip and for cutting out hook portions. In particular, it can be provided that the hook strip is placed by the reel tools being appropriately spaced apart from one another as they forcibly guide the strips. Furthermore, it can be provided that a reel tool is designed to impart a rotary movement for cutting into and/or cutting through the hook strip at the edge of the hook portions to be formed. Reel tools represent a particularly reliable and cost-effective type of processing.

A development of the production device in which the joining unit comprises at least one tool reel and at least one stop reel is more preferable. The at least one tool reel is typically designed for placing, in particular, pressing, the hook strip against the backing layer strip. Preferably, one tool reel, in particular, the same tool reel, is designed to cut the hook portions out of the hook strip. A tool reel designed for attaching the strips and cutting out the hook portions is particularly suitable for simultaneously carrying out method steps b) and c) of the production method described above and below. Typically, the stop reel is designed to form a stop, by means of which the processing forces applied to the hook strip and the backing layer strip by the tool reel are dissipated. This ensures that the method steps are reliably carried out.

Additionally preferred is a development of the production device in which the tool reel has at least one punching projection, wherein the punching projection has a projection height that protrudes radially beyond a reel main body of the tool reel. The projection height preferably corresponds to the thickness of the hook strip. By forming a punching projection on the tool reel, the hook portions can be cut out of the hook strip precisely and quickly. Preferably, the tool reel has multiple punching projections. Typically, the punching projections form a projection contour, in particular, a circumferential projection contour, which corresponds in shape and design to the hook portion to be cut out. The hook portion can then be cut out by rolling the punching projection over the hook strip, whereby the punching projection causes the hook strip to sever or weaken in the region of impact.

The object addressed by the invention is further achieved by a diaper.

The diaper is designed to be placed on a human wearer. The diaper has a diaper main body typically configured to receive, absorb and/or retain media excreted from the wearer's body.

The diaper main body is also typically shaped or designed to be worn in the wearer’s crotch region. The diaper main body has at least one, in particular, two, adhesive portions and at least one, in particular, two, closure tabs. The at least one closure tab, in particular, all the closure tabs, is/are preferably designed to close the diaper in the wearer’s pelvic region and/or hip region.

The at least one closure tab is produced in particular, according to the production method described above and below. This makes it possible to provide a cost-effective diaper that is very comfortable to wear.

The closure tab has at least one textile or textile-like backing layer. The backing layer is typically produced by cutting up or separating the backing layer strip of the closure tab strip. The backing layer is permanently fastened to the diaper main body. Typically, the backing layer is glued to the diaper main body.

The backing layer has a constant backing layer strip thickness. In other words, the backing layer has a constant thickness and preferably has no regions of thinner material. This can guarantee uniform strength of the closure tab.

In addition, the closure tab has at least one hook portion for a detachable hook-and-loop connection to the adhesive portion of the diaper main body. By forming closure tabs having at least one hook portion, the hook-and-loop connection can be a particularly easy connection.

The at least one hook portion of the closure tab has a minimum distance from the closure tab edges of the backing layer on all sides. In other words, the hook portions are set back from the edge of the closure tab. This can prevent the formation of a hard and/or sharp-edged closure tab edge, which reduces the risk of injury when putting on and/or taking off the diaper and increases wear comfort of the diaper.

An embodiment of the diaper in which the closure tab has at least two hook portions for detachably connecting to the adhesive portion of the diaper main body by means of hook-and-loop is preferable. By forming closure tabs having two or more hook portions, the retaining force of the hook-and-loop connection can be adjusted particularly flexibly. In addition, the closure tab can be made more elastic by forming two or more hook portions, thereby improving handling of the closure tab.

Typically, each of the hook portions has a minimum distance from the closure tab edges of the backing layer the entire way around. This can further prevent the formation of a hard and/or sharp-edged edge of the closure tab.

In a preferred embodiment of the diaper, the closure tab has a further backing layer. A further backing layer can adapt the dimensions of the closure tab to diaper-specific specifications without having to change the backing layer and the hook portions. This makes it particularly easy to adjust the closure tab. In addition, a further backing layer can increase the strength of the closure tab in region where it overlaps with the backing layer, which can make the closure tab more durable.

The further backing layer protrudes beyond the at least one backing layer, at least in portions. The further backing layer protrudes beyond the backing layer at least on one side. Typically, the further backing layer, in particular, the portion of the further backing layer protruding beyond the backing layer, is designed for permanently fastening the closure tab to the diaper main body. In other words, the closure tab forms a fastening portion.

Preferably, the further backing layer protrudes beyond the backing layer at least on either side of the hook portions. Further preferably, the further backing layer forms a fastening portion facing the diaper main body and an actuating portion facing away from the diaper main body. The actuating portion and the fastening portion are preferably formed on opposite sides of the hook portions. An actuating portion simplifies handling of the closure tab when establishing and releasing the hook-and-loop connection.

A development of the diaper in which the at least one backing layer and the further backing layer of the closure tab collectively form at least one, in particular at least two, portions of the closure tab edges is also preferred. This allows the edges of the closure tab to be more rigid but prevents them from being hard or sharp.

In a preferred embodiment of the diaper, at least one of the backing layers of the closure tab is rectangular, trapezoidal or parabolic. By designing the closure tab accordingly, it can be easily adapted to different types of diapers. In addition, handling of the closure tab can be improved.

An embodiment of the diaper in which the closure tab has at least three hook portions arranged next to one another in a line is also preferred. Three or more hook portions allow for a particularly secure hook-and-loop connection, especially if the hook regions do not completely overlap an adhesive region of the diaper. This can ensure that at least one hook portion creates a hook-and-loop connection with the adhesive portion of the diaper.

A preferred development of the diaper provides that the hook portions of the closure tab are identical. This allows for particularly even distribution of the retaining forces of the hook-and-loop connection across the individual hook portions.

In a preferred embodiment of the diaper, the hook portions of the closure tab are rectangular or circular, in particular elliptical. Rectangular or circular hook portions simplifies the production of the closure tab and reduces production costs. In addition, rectangular or circular hook portions has proven to be particularly suitable for creating a secure hook-and-loop connection.

An embodiment of the diaper in which the closure tab is designed to create a hook-and-loop connection between the at least one hook portion and the adhesive portion directly on the wearer’s hip when the wearer is wearing the diaper is also preferred. Preferably, the diaper has two or more closure tabs. In other words, the closure tab, in particular, the closure tabs, is/are designed for use on a so-called pull-on diaper or diaper pants. The production method described above and below has proven to be particularly suitable for the production of closure tabs for such diapers.

A development of the diaper is also preferred in which the at least one hook portion and the at least one adhesive portion extend transversely or perpendicularly to a closure direction of the diaper. In particular, when using such closure tabs or hook portions on pull-on diapers that are closed at the sides, a secure hook-and-loop connection in conjunction with easy manual release of the hook-and-loop connection can be achieved by correspondingly arranging the hook portions and adhesive portions.

Further advantages of the invention can be found in the description and the drawings. Likewise, according to the invention, the aforementioned features and those which are to be explained below can each be used individually or together in any desired combinations. The embodiments shown and described are not to be understood

as an exhaustive list, but, rather, have an exemplary character for the description of the invention.

### BRIEF DESCRIPTION OF THE DRAWINGS

FIG. 1 shows a schematic view of a production method for producing a closure tab.

FIG. 2 shows a schematic view of a first embodiment of a production device for producing a closure tab, having a supply unit, a joining unit and a removal unit.

FIG. 3 shows a second embodiment of a production device for producing a closure tab having a supply unit having three supply reel.

FIG. 4 shows a partially sectional view of a joining unit having a tool reel, a stop reel and multiple strips arranged therebetween.

FIG. 5 shows a schematic view of a closure tab strip.

FIG. 6 shows a schematic view of a first embodiment of a closure tab.

FIG. 7 shows a perspective view of a first embodiment of a diaper comprising a diaper main body and two closure tabs according to a second embodiment.

FIG. 8 shows a perspective view of a second embodiment of a diaper comprising a closure tab that can be closed directly on a wearer's hip according to a third embodiment.

### DETAILED DESCRIPTION OF THE PREFERRED EMBODIMENTS

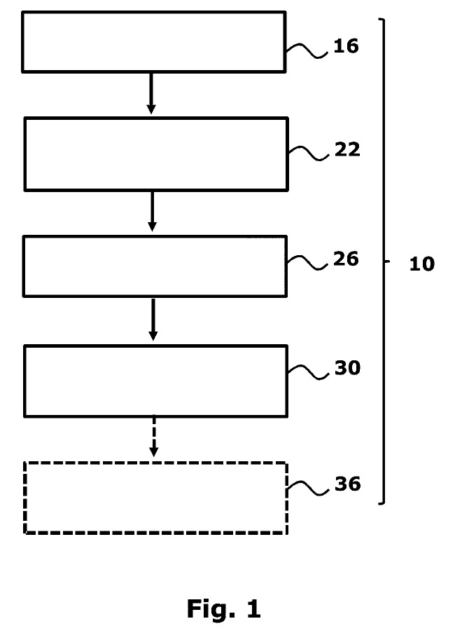

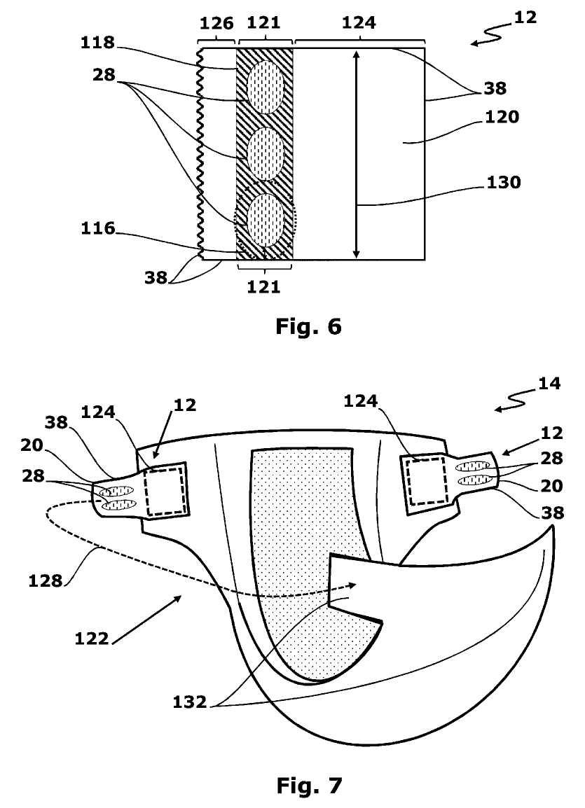

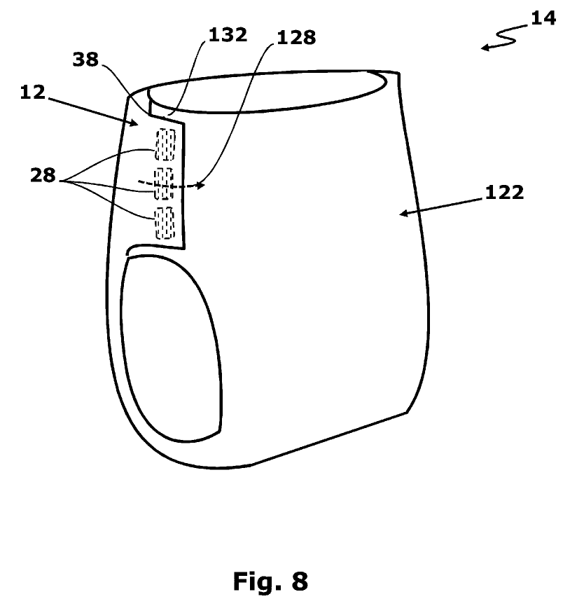

FIG. 1 shows a schematic view of a production method 10 according to the invention. The production method 10 is intended for producing a closure tab 12 (see FIG. 6-8) for a diaper 14 (see FIG. 7 and 8).

The production method 10 will be explained in the following with reference to the other figures in the drawings.

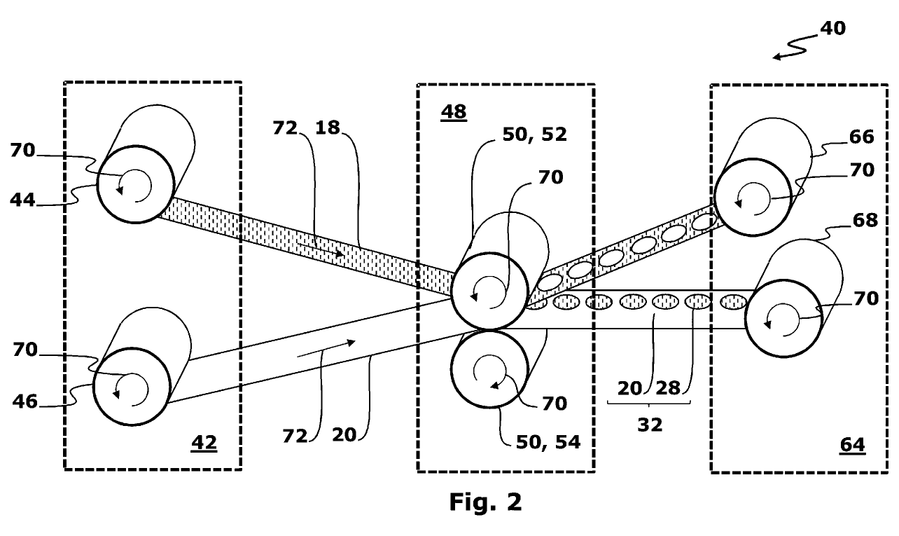

A first method step 16 of the production method 10 provides the provision of a hook strip 18 (see FIG. 2-4) and at least one backing layer strip 20 (see FIG. 2-4). The hook strip 18 is typically designed as a hook-and-loop strip having hooks (not shown) and/or mushrooms (not shown). The backing layer strip 20 is typically a textile or textile-like backing layer strip, preferably in the form of a nonwoven.

The hook strip 18 and the backing layer strip 20 are preferably designed to be unrolled and rolled up. This enables the hook strip 18 and the backing layer strip 20 to be provided as rolls, whereby the production method 10 can be carried out particularly effectively. Preferably, the hook strip 18 and the backing layer strip 20 are provided on separate reels. This can simplify the preparation process for the joining method 10.

A subsequent method step 22 of the production method 10 provides placing the hook strip 18 on the backing layer strip 20. Placing the strips against one another can be understood to mean establishing surface contact between the hook strip 18 and the backing layer strip 20. Preferably, the hook strip 18 is pressed on the backing layer strip 20. This can ensure they abut in particular so as to be surface-to-surface. In addition, process-related unevenness in the hook strip 18 and/or the backing layer strip 20 can be smoothed out by pressing, which simplifies further processing of the strips that are placed against one another.

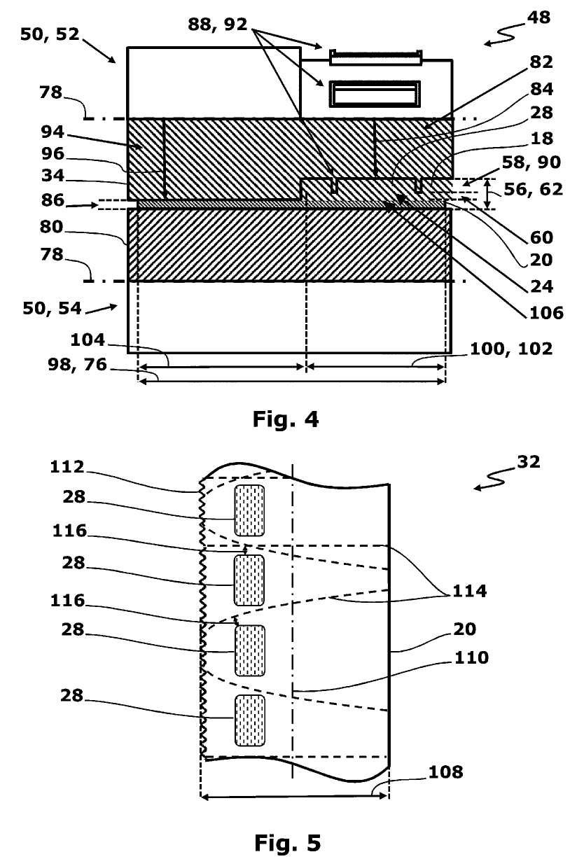

Typically, the hook strip 18 is fastened to the backing layer strip 20, at least in portions, when placed thereon. For this purpose, a binder (not shown), in particular an adhesive, may be introduced into a contact region 24 (see FIG. 4) between the hook strip 18 and the backing strip 20. Alternatively or additionally, it can be provided that the portions of the hook strip 18 are welded to the backing layer strip 20.

A further method step 26 of the production method 10 provides cutting at least one hook portion 28 (see FIG. 2-8) out of the hook strip 18. Typically, in method step 26, multiple, in particular a large number of, hook portions 28 are cut out of the hook strip 18. In other words, method step 26 can be repeated more than once or several times.

“Cutting out” can be understood to mean separating the material bond between the hook portion 28 to be cut out and the remaining hook strip 18. Cutting out can also occur due to weakening of the material bond if, as a result of weakening said bond, the retaining forces between the backing layer strip 20 and the hook strip 18 exceed the retaining forces between the hook portion 28 to be cut out and the remaining hook strip 18. Preferably, the hook strip 18 is fastened to the backing layer strip 20, preferably by introducing binder or by welding, only in the contact region 24 between a hook portion 28 to be cut out and the backing layer strip 20. As a result, the retaining forces between the hook portion 28 to be cut out and the backing layer strip 20 can be increased compared to those between the hook portion and the remaining hook strip 18.

Said hook portion is preferably mechanically cut out by cutting or cutting into and/or punching the hook strip 18.

According to the invention, the at least one hook portion 28 is cut out the hook strip 18 without damaging the backing layer strip 20. This allows for further processing of the strips placed against one another without the risk of the strips breaking.

According to the invention, a method step 30 provided, in which a closure tab strip 32 (see FIG. 2, 3 and 5) is created by detaching the hook strip 18 from the at least one backing layer strip 20. When detaching the hook strip 18, the at least one cut-out hook portion 28 remains on the backing layer strip 20 and forms the closure tab strip 32 against the backing layer strip 20.

If the hook strip 18 is fastened to the backing layer strip 20, the hook strip 18 is preferably detached from the backing layer strip 20 before the full retaining force of the fastening has developed, for example before a binder and/or a weld seam hardens. Alternatively, it can be provided that fastening takes place only in the contact region of the at least one hook portion 28 to be cut out. This allows the hook strip to be detached once the retaining force has fully developed, thereby increasing the process reliability of the production method 10.

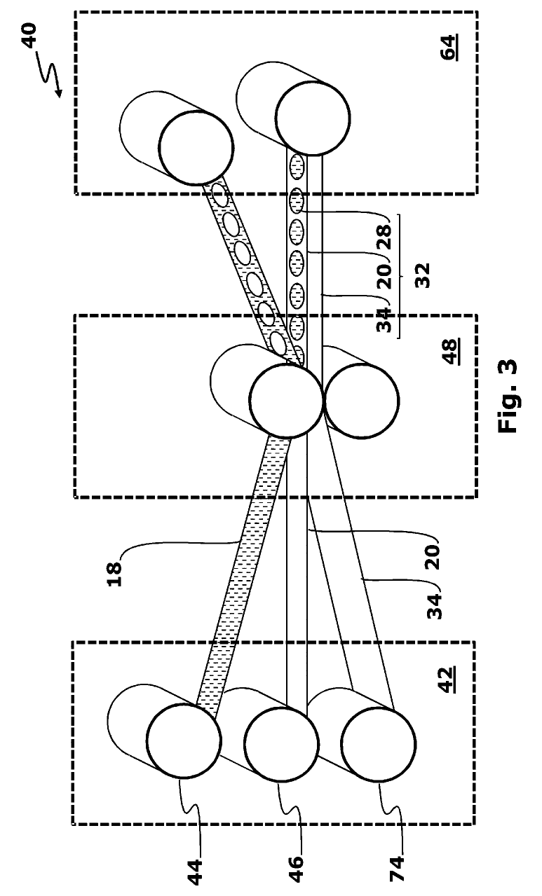

In a particular embodiment of the production method 10, it is provided that in step 16 a further backing layer strip 34 (see FIG. 3 and 4) is provided. The production method 10 can further provide in method step 22 that the backing layer strip 20 is fastened to the further backing layer strip 34. The backing layer strip 20 is preferably fastened to the further backing layer strip 34 by introducing a binder (not shown). Typically, surface contact is formed between the backing layer strip 20 and the further backing layer strip 34 by pressing. A further backing layer strip 34 increases the strength of the closure tab strip 32 and thus the strength of the closure tabs 12 to be produced.

In a particularly preferred embodiment, the production method 10 has a method step 36 which provides the creation of closure tabs 12 by forming closure tab edges 38 (see FIG. 6—8) by removing a portion of the closure tab strip 32. Preferably, the portion is mechanically removed by cutting and/or punching the closure tab strip 32.

The creation of the closure tabs 12 can directly follow the creation of the closure tab strip 32. Typically, the portion is removed by means of a subsequent production method, wherein a closure tab strip 32 is provided, preferably rolled up.

In a particularly preferred embodiment of the production method 10, the method steps 22, 26 are carried out simultaneously. In other words, the backing layer strips 20, 34 and the hook strip 18 are placed against one another and the hook portions 28 are cut out of the hook strip 18 simultaneously. This allows the production method 10 to be carried out particularly robustly along with a simple design of a production device 40 (see FIG. 2 and 3).

FIG. 2 shows a production device 40 suitable for producing a closure tab 12 (see FIG. 6-8). The production device 40 is typically configured to carry out the production method 10 (see FIG. 1).

The production device 40 has a supply unit 42 for providing a hook strip 18 and a backing layer strip 20. The supply unit 42 can, for example, be formed as a reel magazine (not shown in detail). As shown, the supply unit 42 can have a first supply reel 44 for providing a rolled-up hook strip 18 and a second supply reel 46 for providing a rolled-up backing layer strip 20. Typically, the supply reels 44, 46 are designed to hold several hundred meters of strip. By rotating the supply reels 44, 46, the hook strip 18 or backing layer strip 20 is unrolled and provided for the production method 10.

The production device 40 further has a joining unit 48. The joining unit 48 is designed to attach the hook strip 18 to the backing layer strip 20. As shown, the joining unit 48 can comprise reel tools 50—in this case a tool reel 52 and a stop reel 54—between which the hook strip 18 and the backing layer strip 20 are guided.

The radial tool reel distance 56 (see FIG. 4) between the tool reel 52 and the stop reel 54 preferably corresponds to the total material thickness 62 resulting from the hook strip thickness 58 (see FIG. 4) of the hook strip 18 and the backing layer strip thickness 60 (see FIG. 4) of the backing layer strip 20 (see FIG. 4). In a particularly preferred embodiment, the radial tool reel distance 56 is slightly smaller than the total material thickness 62, and therefore the hook strip 18 can be pressed against the backing layer strip 20.

The joining unit 48 is also designed to cut the hook portions 28 out of the hook strip 18. As shown, the tool reel 52 of the joining unit 48 can be designed to cut—in this case punch—the hook portions 28 out of the remaining hook strip 18.

In addition, the production device 40 comprises a removal unit 64. The removal unit 64 is designed to receive the hook strip 18 and the closure tab strip 32 separately. The removal unit 64 can have a reel magazine (not shown in detail). As shown, the removal unit 64 has a first removal reel 66 for receiving the hook strip 18 and a second removal reel 68 for receiving the closure tab strip 32. Typically, the removal reels 66, 68 are designed to roll up the corresponding strip.

To operate the production device 40, it is typically provided that the hook strip 18 and the backing layer strip 20 are partially unrolled from the rolled-up state of the corresponding supply reel 44, 46, guided between the working reels 50 of the joining unit 48 and fastened to the corresponding removal reel 66, 68.

Subsequently, one or more of the reels 44, 46, 52, 54, 66, 68 can be set in a rotary movement 70. Preferably, one of the reels 44, 46, 52, 54, 66, 68, particularly preferably the reels 50 of the joining unit 48 and/or the reels 66, 68 of the removal unit 64 has a reel drive (not shown in detail). The non-driven reels can be forced to rotate by the arrangement of the hook strip 18 and the backing layer strip 20.

During operation of the production device 40, the hook strip 18 and the backing layer strip 20 are continuously unrolled in the feed directions 72 of the production device 40 from the corresponding supply reel 44, 46, placed against one another in the joining unit 48, in particular pressed together, the hook portions 48 are cut out of the hook strip 18, the hook strip 18 is detached from the backing layer strip 20, or lifted off, and then rolled up on the corresponding removal reel 66, 68. The resulting closure tab strip 32 can then be further processed to produce closure tabs 12. This enables the particularly fast, robust and cost-effective production of closure tabs 12 that have a high level of wearing comfort.

FIG. 3 shows a particular embodiment of the production device 40.

As shown, the production device 40 comprises, in addition to the first supply reel 44 and the second supply reel 46, a third supply reel 74 for holding the further backing layer strip 34. This enables the production method 10 according to the invention (see FIG. 1) to be carried out while providing the further backing layer strip 34.

Using the further backing layer strip 34, the production method 10 can be carried out substantially analogously to the previously described procedure, wherein multiple strips, specifically the hook strip 18, the backing layer strip 20 and the further backing layer strip 34, are placed against one another in the joining unit 48. In other words, the production device 40 is designed for placing, preferably pressing, at least three strips against one another. The closure tab strip 32 has, as shown, the backing layer strip 20, the further backing layer strip 34 and the hook portions 28.

By using a further backing layer strip 34, the closure tab strip 32 can be modified in terms of a closure tab strip width 76 (see FIG. 4) and the total material thickness 62, in particular in the region of the hook portions 28, can be increased. This allows the closure tab 12 to be adapted for use with a specific type of diaper 14 (see FIG. 7 and 8). Furthermore, the resistance of the closure tabs 12 produced from the closure tab strip 32 can be increased.

The removal unit 64 can remain unchanged, thereby minimizing the costs of providing and modifying the production device 40 and enabling easy adaptation to different designs of the closure tab strip 32.

FIG. 4 schematically shows an example joining unit 48 with a hook strip 18, a backing layer strip 20 and a further backing layer strip 34 arranged therebetween.

As shown, the joining unit 48 is designed for placing the strips against one another and for cutting the hook portions 28 out of the hook strip 18.

The joining unit 48 has, as shown, two tool reels 50—in this case the tool reel 52 and the stop reel 54—which are each mounted, in particular, driven, so as to be rotatable about an axis of rotation 78. The tool reels 50 are designed to dissipate the forces generated during joining of the strips of when placing them against one another.

The stop reel 54 preferably has a constant reel radius 80. This allows the production costs for the stop reel 54 to be minimized.

The tool reel 52 can have a reel main body 82 with a main body radius 84. The reel main body 82 preferably forms the tool reel distance 56 together with the stop reel 54. The tool reel distance 56 typically forms a nominal size of the total material thickness 62 of the closure tab strip 32 assembled from the individual strips. In other words, the tool reel distance 56 can be the sum of the hook strip thickness 58, the backing layer strip thickness 60 and, if applicable, a strip thickness 86 of the further backing layer strip 34.

The backing layer strip thickness 60 of the backing layer strip 20 is preferably constant. In other words, the material thickness 58 of the backing layer strip 20 does not change. As a result, the strength of the backing layer strip 20 and thus of the closure tab strip 32 remains constant. In addition, the production method 10 and the reel tools 50 can be kept simple, thereby minimizing costs.

Preferably, the strip thickness 86 of the further backing layer strip 34 and/or the hook strip thickness 58 is/are also constant. This can enhance the advantages described above.

In a particular embodiment, the tool reel distance 56 can be smaller than the total material thickness 62 of the closure tab strip 32. This can cause the strips to be pressed together in the joining unit 48, thereby increasing the hold between the strips.

As shown, the tool reel 52 can have at least one, here multiple, punching projections 88. The punching projections 88 can be arranged or formed on the tool reel 52. Preferably, the punching projections 88 are arranged or formed so as to be evenly distributed over the circumference of the tool reel 52. The punching projections 88 protrude in the radial direction of the tool reel 52 beyond the reel main body 82 by a projection height 90 and are designed to cut the hook portions 28 out of the hook strip 18. Typically, the projection height 90 is the size of the hook strip thickness 58. This can prevent the backing layer strip 20 from being damaged when the hook portions 28 are cut out.

In a particular embodiment, the projection height 90 can be smaller than the hook strip thickness 58 in order to take into account any compression of the hook strip 18 when the strips are pressed together in the joining unit 48. The projection height 90 preferably corresponds to the hook strip thickness 58 when the hook strip 18 is compressed.

In a particularly preferred embodiment, the projection height 90 can be smaller than the hook strip thickness 58 when the hook strip 18 is compressed. As a result, the hook strip 18 is not completely cut through, which means that damage to the backing layer strip 20 can be prevented even more reliably. The part of the hook strip 18 that is not cut through has lower retaining forces with respect to the hook portion 28 than the backing layer strip 20 has with respect to the hook portion 28. When the hook strip 18 is detached from the backing layer strip 20, the hook portion 28 can be torn off from the hook strip 18.

Preferably, the retaining forces between the hook portion 28 and the backing layer strip 20 are formed by introducing a binder (not shown) and/or by welding the hook portion 28 to the backing layer strip 20 in the contact region 24. This makes the production behavior 10 more robust and less susceptible to errors.

The punching projections 88 are preferably designed as cutting edges. This allows the hook portions 28 to be cut out using little force.

The punching projections 88 have a separating contour 92. The separating contour 92 is adapted to the hook portion 28 to be cut out. As shown, the separating contour 92 is rectangular. However, other separating contours 92 are also possible, for example circular, elliptical, triangular or polygonal separating contours 92. The separating contours 92 can also have an angular basic design, but with rounded corners.

Typically, the punching projections 88 of a tool reel 52 are identical, and therefore the tool reel 52 can be manufactured particularly cost-effectively. However, in order to form different-shaped hook portions 28, it can also be provided that the punching projections 88 have different separating contours 92. The different separating contours 92 can be arranged or formed in a predetermined order on the tool reel 52.

As shown, the tool reel 52 can have a support portion 94 projecting radially beyond the reel main body 82. The support portion 94 therefore has a support radius 96 which is larger than the main body radius 84. Typically, the support portion 94 projects beyond the punching projections 88 by the backing layer strip thickness 60 when the backing layer strip 20 is not compressed.

Typically, the support portion 94 has a radial distance from the stop reel 54 which corresponds to the strip thickness 86 of the further backing layer strip 34 or the sum of the strip thickness 86 and the backing layer strip thickness 60 of the backing layer strip 20.

The support portion 94 can thus be used to guide the further backing layer strip 34 and/or the backing layer strip 20 when a strip width 98 of the further backing layer strip 34 and/or a backing layer strip width 100 of the backing layer strip 20 exceeds a hook strip width 102.

As shown, the support portion 94 guides the further backing layer strip 34 in a portion protruding beyond the hook strip 18 and the backing layer strip 20 by the overhang 104.

To form the closure tab strip 32 (see FIG. 2, 3 and 5), it can be provided that before the backing layer strip 20 and the further backing layer strip 34 are placed against one another, a binder is introduced into the contact region 106 between the backing layer strip 20 and the further backing layer strip 34. This can create a particularly rigid connection between the strips.

As shown, the resulting closure tab strip 32 has a closure tab strip width 76 which corresponds to the strip width 98 of the further backing layer strip 34.

FIG. 5 is a schematic cut-out of an example closure tab strip 32.

The closure tab strip 32 has the backing layer strip 20 and multiple hook portions 28. The hook portions 28 are identical and are arranged or fastened along a strip main extension 110 one behind the other in a row on the backing layer strip 20. The hook portions 28 are at equal distances from one another or have the same distance from the adjacent hook portion 28 each time. The strip main extension 110 runs perpendicularly to the closure strip width 76.

As shown, the closure tab strip 32, or the backing layer strip 20 and/or the further backing layer strip 34 (see FIG. 3, 4 and 6) can have a pre-shaped strip edge 112 which is jagged here.

The closure tab strip 32 is suitable for producing closure tabs 12. For this purpose, the closure tab strip 32 can be cut along separating lines 114, thereby forming the closure tab edges 38 (see FIG. 6-8). The separating lines 114 can be straight and/or curved, as shown. In addition, angular progressions of the separating lines 114 are conceivable.

Preferably, when cutting the closure tab strip 32, a minimum distance 116 from the separating line 114 to the corresponding hook portion(s) 28 is maintained. In other words, the hook portion 28 does not form part of the closure tab edge 38 of the closure tab 12 to be produced (see FIG. 6-8).

FIG. 6 shows an example closure tab 12.

The closure tab 12 is formed from a closure tab strip 32 (see FIG. 25) and has a textile or textile-like backing layer 118, a textile or textile-like additional backing layer 120 as well as—in this case multiple—hook portions 28. Preferably, the backing layer 118 and/or the further backing layer 120 is/are formed as a nonwoven material.

The backing layer 118 has a constant backing layer strip thickness 60 (see FIG. 4). In other words, a cross section of the backing layer 118 is not weakened. This ensures particularly uniform strength of the closure tab 12. As shown, multiple hook portions 28 are fastened to the backing layer 118.

The backing layer 118 and the further backing layer 120 extend between closure tab edges 38, at least some of which are formed by cutting the backing layer 20 (see FIG. 2-5) and the further backing layer 34 (see FIG. 3 and 4) of a closure tab strip 32 at—in this case straight—separating lines 114 (see FIG. 5).

As shown, the backing layer 118 and the further backing layer 120 of the closure tab 12 collectively form a portion 121 of two closure tab edges 38 in each case. The portions 121 have increased strength, thereby improving the handling of the closure tab 12.

The hook portions 28 are arranged on or fastened to the backing layer 118 and have a circumferential minimum distance 116 from the closure tab edges 38. As a result, the hook portions 28 are set back behind the closure edges 38, and therefore the closure edges 38 can be soft and the wear comfort of the diaper 14 is increased.

As shown, the hook portions 28 are elliptical and are designed for detachably fastening the closure tab 12 to a diaper main body 122 (see FIG. 7 and 8) of a diaper 14 (see FIG. 7 and 8). Typically, the hook portions 28 form a hook-and-loop connection with the diaper main body 122.

The closure tab 12 has, as shown, a fastening portion 124 for attaching the closure tab 12 to the diaper main body 122 and an actuating portion 126 for actuating the closure tab 12. The fastening portion 124 and the actuating portion 126 are formed here by the further backing layer 120. In other words, the further backing layer 120 protrudes, at least in portions, beyond the at least one backing layer 118. The actuating portion 126 is preferably formed on a side of the hook portions 28 opposite the fastening portion 124. This makes it easier to actuate the actuating portion 124. When the closure tab 12 is fastened to the diaper main body 122, the actuating portion 124 is flexible or not connected to the diaper main body 122 such that the actuating portion 126 can be gripped and the fastening by means of the hook portions 28 can be released more easily.

The fastening portion 124 serves to fasten, in particular, to glue, the closure tab 12 to the diaper main body 122. The extension of the fastening portion 124 can be adapted to the type of diaper 14.

The fastening portion 124 is preferably at least the same size as the hook strip 102, particularly preferably is twice the hook strip width 102, in a closure direction 128 (see FIG. 7 and 8). This allows a particularly secure fastening of the closure tab 12 to the diaper main body 122.

The actuating portion 126 is at least a third of the hook strip width 102, particularly preferably is the hook strip width 102, in the closure direction 128. This ensures simple and quick actuation.

The closure tab 12 can, as shown, have a closure tab length 130. The closure tab length 130 is preferably at least the same size as the closure tab strip width 76, particularly preferably at least twice the closure tab strip width 76.

FIG. 7 shows a diaper type by way of example.

When not being worn, the diaper 14 has a diaper main body 122 and two closure tabs 12. When not being worn, the closure tabs 12 are not attached to an adhesive portion 132 (so-called “target area”) of the diaper main body 122 by means of the—in this case two—hook portions 28, thereby making it easier to put the diaper 14 on a human wearer (not shown).

When the wearer puts the diaper 14 on, the closure tabs 12 can be placed on the corresponding adhesive portion 132 of the diaper main body 122, thereby creating a hook-and-loop connection between the hook portions 28 and the corresponding adhesive portion 132. This allows the diaper to be securely held in place when being worn. The attachment of the closure tab 12 is shown by the closure direction 128 (dashed arrow). As shown, the hook portion 28 extends in the closure direction 128, whereby the retaining force of the hook portion 28 to the adhesive portion 132 is increased.

To remove the diaper 14, the hook-and-loop connection formed by the hook portion 28 and the adhesive portion 132 can be released by pulling the hook portion 28 off the adhesive portion 132.

The hook portions 28 are preferably set back behind the corresponding closure edge 38 by the minimum distance 116 (see FIG. 5 and 6). As a result, there is no hard and/or sharp closure edge 38 caused by the hook portion 28, which increases the wear comfort of the diaper 14 and reduces the risk of injury when putting it on and taking it off.

The closure tabs 12 are arranged on or fastened, in particular glued, to the diaper main body 122 by means of the corresponding fastening portion 124.

The type of diaper 14 illustrated is designed to be closed in the pelvic region of a wearer. This allows the diaper 14 to be easily put on from a frontal direction by a person entrusted with putting on the diaper 14.

FIG. 8 shows another example type of diaper 14.

The diaper 14 has a diaper main body 122 and two closure tabs 12, with the representation only showing one of the closure tabs 12. As shown, each closure tab 12 has three hook portions 28.

Typically, the illustrated type of diaper 14 is put on when closed like pants by inserting the wearer’s legs (not shown). This makes it possible to put on the diaper 14 particularly easily and quickly, especially while the wearer is standing. In addition, the wearer can pull the diaper 14 down and back up themselves, especially when the wearer is “cleaning” themselves up.

To remove the diaper 14, the closure tabs 12 can be released by detaching the hook portions 28 from the corresponding adhesive portion 132 and the diaper main body 122 can be opened. This allows for hygienic removal of the diaper 14 without having to pass the legs through the used diaper 14.

As shown, the closure tabs 12 are designed, when the wearer is wearing the diaper 14, for connecting the—in this case three—hook portions 28 of each closure tab 12 to the corresponding adhesive portion 132 directly on the wearer’s hip (not shown) by means of hook-and-loop. This allows the diaper 14 to be opened particularly hygienically.

The hook portions 28 of the closure tabs 12 extend transversely to the closure direction 128 of the diaper 14. This allows for wide fastening and the diaper 14 can thus be securely worn and securely held in position at the wearer's hips.

The hook portions 28 set back behind the closure edges 38 render the diaper 14 very comfortable to wear and allow for a low risk of injury to the wearer when putting the diaper 14 on and taking it off.

Taking an overview of all the figures in the drawings, the invention relates to a production method 10 for producing a closure tab strip 32 for creating closure tabs 12 for a diaper 14. The production method 10 provides the surface-to-surface placement of two or more strips against of one another, wherein at least one of the strips is designed as a hook strip 18. Preferably, when placing the strips against one another, hook portions 48 are punched out of the hook strip 18 and fastened to a backing layer strip 20. The closure tab strip 32 produced can then be fed for further method steps for forming the closure tab 12. The invention also relates to a production device 40 which enables the closure tab 32 to be produced according to the invention. The invention further relates to a diaper 14 which can be produced particularly inexpensively and, moreover, ensures high wear comfort with a low risk of injury.

### List of reference signs:

10 production method;

12 closure tab;

14 diaper;

16 method step;

18 hook strip;

20 backing layer strip;

22 method step;

24 contact region;

26 method step;

28 hook portion;

30 method step;

32 closure tab strip;

34 further backing layer strip;

36 method step;

38 closure tab edge;

40 production device;

42 supply unit;

44 first supply reel;

46 second supply reel;

48 joining unit;

50 reel tools;

52 tool reel;

54 stop reel;

56 tool reel distance;

58 hook strip thickness;

60 backing layer strip thickness;

62 total material thickness;

64 removal unit;

66 first removal reel;

68 second removal reel;

70 rotary movement;

72 feed direction;

74 third supply reel;

76 closure tab strip width;

78 axis of rotation;

80 reel radius;

82 reel main body;

84 main body radius;

86 strip thickness;

88 punching projection;

90 projection height;

92 separating contour;

94 support portion;

96 support radius;

98 strip width;

100 backing layer strip width;

102 hook strip width;

104 overhang;

106 contact region;

110 strip main extension;

112 strip edge;

114 separating line;

116 minimum distance;

118 backing layer;

120 further backing layer;

121 portion;

122 diaper main body;

124 fastening portion;

126 actuating portion;

128 closure direction;

130 closure tab length;

132 adhesive portion.

What is claimed is:

1. A production method for producing a closure tab for a diaper, having the method steps of:
a) providing a hook strip and at least one textile or textile-like backing layer strip;
b) placing the hook strip and the backing layer strip against one another and fastening the hook strip to the backing layer strip at least in portions;
c) cutting at least one hook portion out of the hook strip;
d) producing a closure tab strip by partially detaching the hook strip from the at least one backing layer strip, wherein the at least one cut-out hook portion remains on the backing layer strip.

2. The production method according to claim 1, wherein, in method step a), a further backing layer strip is provided; wherein, in method step b), the backing layer strip is fastened to the further backing layer strip.

3. The production method according to claim 1, additionally having the method step of:
e) producing closure tabs by forming closure tab edges by removing a portion of the closure tab strip, wherein each closure tab edge is formed at a minimum distance from the at least one hook portion.

4. The production method according to claim 1, wherein method step b) and method step c) are carried out simultaneously.

5. A production device for producing a closure tab, designed to carry out the production method according to claim 1, having: a supply unit for providing a hook strip and a backing layer strip, a joining unit for joining the hook strip to the backing layer strip, and a removal unit for separately receiving the hook strip and a closure tab strip; wherein the joining unit is designed for placing, in particular pressing, the hook strip on the backing layer strip and for cutting hook portions out of the hook strip.

6. The production device according to claim 5, wherein the joining unit comprises reel tools between which the hook strip and the backing layer strip are guided; wherein the reel tools are designed and positioned relative to one another so as to place or press the hook strip and to cut out the hook portions.

7. The production device according to claim 6, wherein the joining unit comprises at least one tool reel and at least one stop reel, wherein the at least one tool reel is designed for pressing the hook strip and for cutting out the hook portions.

8. The production device according to claim 7, wherein the tool reel has at least one punching projection, wherein the punching projection has a projection height projecting radially beyond a reel main body of the tool reel, wherein the projection height corresponds to a hook strip thickness of the hook strip.

9. A diaper for arrangement on a wearer, having a diaper main body, at least one adhesive portion and at least one closure tab, wherein the at least one closure tab is produced as per the production method according to claim 3; wherein the closure tab has at least one textile or textile-like backing layer and is permanently fastened to the diaper main body; wherein the closure tab has at least one hook portion for releasably connecting to the adhesive portion by means of hook-and-loop; wherein the at least one backing layer has a constant backing layer strip thickness; and wherein the at least one hook portion has a minimum distance from the closure tab edges of the backing layer on all sides.

10. The diaper according to claim 9, wherein the closure tab has at least two hook portions for releasably connecting to the adhesive portion by means of hook-and-loop.

11. The diaper according to claim 9, wherein the closure tab has a further backing layer, wherein the further backing layer protrudes at least in portions beyond the at least one backing layer and is designed for permanently fastening the closure tab to the diaper main body.

12. The diaper according to claim 11, wherein the at least one backing layer and the further backing layer of the closure tab collectively form at least one portion of the closure tab edges.

13. The diaper according to claim 9, wherein at least one of the backing layers of the closure tab is rectangular, trapezoidal or parabolic.

14. The diaper according to claim 9, wherein the closure tab has at least three hook portions arranged next to one another in a line.

15. The diaper according to claim 14, wherein the hook portions of the closure tab are identical.

16. The diaper according to claim 9, wherein the hook portions of the closure tab are rectangular, circular or elliptical.

17. The diaper according to claim 9, wherein, when the wearer is wearing the diaper, the closure tab is designed to create a hook-and-loop connection between the at least one hook portion and the adhesive portion directly on the wearer’s hip.

18. The diaper according to claim 17, wherein the at least one hook portion extends transversely or perpendicularly to a closure direction of the diaper.
